(19) United States
(12) Patent Application Publication
(10) Pub. No.: US 2007/0112379 A1
(43) Pub. Date: May 17, 2007

(54) Device for the removal of ticks from the body of humans or animals

(75) Inventor: Helmut Vaic, Eckental (DE)

Correspondence Address: BROWDY AND NEIMARK, P.L.L.C.;624 NINTH STREET, NW, SUITE 300, WASHINGTON, DC, 20001-5303, US

(73) Assignee: Facklemann GmbH & Co. KG, Hersbruck (DE)

(21) Appl. No.: 11/503,210

(22) Filed: Aug. 14, 2006

(30) Foreign Application Priority Data
Nov. 12, 2005 (DE) 10 2005 054 124.0

(51) Int. Cl. A61B 17/50

(52) U.S. Cl. 606/211

(57) ABSTRACT

In a device for the removal of ticks from the skin of humans and animals comprising a forceps configuration for grasping the tick's body in the region of the skin surface, as well as a reservoir having an actuation means for a freeze spray to be sprayed onto the tick's body prior to its removal, provision is made for a main body to be provided that incorporates a hollow space for accommodating the reservoir for the defrosting [sic] spray, a discharge nozzle for the freeze spray on the underside of the main body, and a forceps configuration that extends around the main body into the region below the discharge nozzle.

### BACKGROUND OF THE INVENTION

1. Field of the Invention

The invention is concerned with a device for the removal of ticks from the skin of humans and animals, comprising a forceps configuration for grasping the tick's body in the region of the skin surface, as well as a reservoir having a valve for a freeze spray to be sprayed onto the tick's body prior to its removal. To the extent that a tick's body is mentioned here, this shall also include so-called nymphs.

2. Background Art

A device of this type is known from DE 203 07 382 U1.

### SUMMARY OF THE INVENTION

The invention is based on the object of improving a device, the generic type of which is assumed to be known, in such a way that it can be produced even more cost effectively and manipulated even more easily.

This object is met according to the invention in such a way that a main body is provided that has a hollow space for accommodating the reservoir for the defrosting [sic] spray, a discharge nozzle for the freeze spray on the underside of the main body, and a forceps configuration extending around the main body into the region below the discharge nozzle.

This design results in a highly compact configuration and permits the main body and forceps configuration to be injection-molded from plastic as one piece, so that the entire device consists only of one injection-molded piece.

In a further improvement of the invention, provision is made for the forceps configuration to have two legs that are symmetrical to each other, each leg comprising an actuation section and a forceps section, each leg being convexly arched outward, and each leg being pivot-mounted in the transition region between the actuation section and forceps section in such a way that when a pulling force is exerted onto the actuation section in an inward direction, the associated forceps section is moved outward. This permits a simple and uncomplicated operation with one hand.

Each pivot bearing is preferably formed at the outer end of a cross brace that is disposed on the underside of the main body, especially in such a way that both ends of the cross brace have bearing openings formed on them that are circular-segment shaped in cross-section, into which a bearing projection of corresponding cross-section of the respective leg can be locked in place that extends away from the inside of the leg in the transition region of the actuation section and forceps section.

The outsides of the actuation sections preferable have a ribbed surface in order to render them non-slipping.

The invention will be explained in more detail below based on a preferred example embodiment, in conjunction with the drawing.

### BRIEF DESCRIPTION OF THE DRAWING

FIG. 1 shows a side view of an inventive device in the initial position, and

FIG. 2 shows an illustration corresponding to FIG. 1 with pressure being exerted laterally in an inward direction.

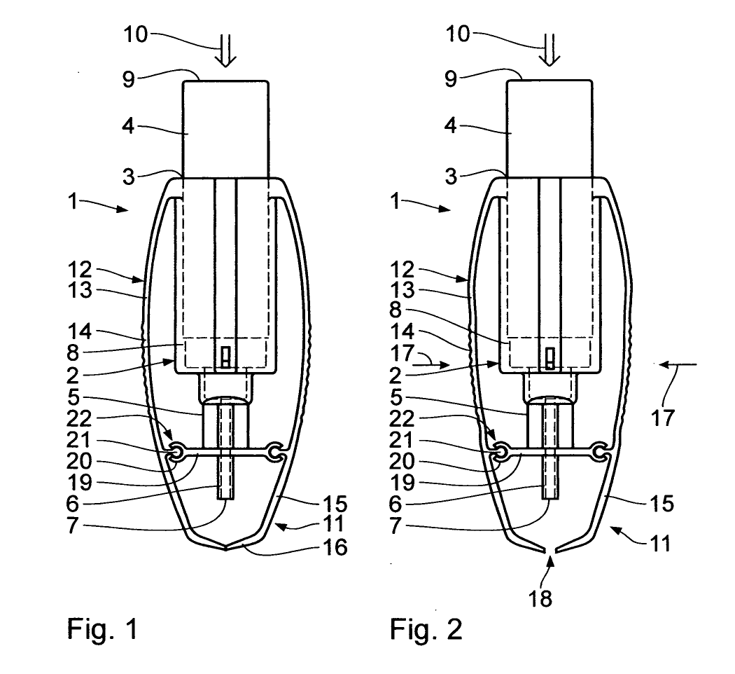

### DESCRIPTION OF THE PREFERRED EMBODIMENT

A device 1 shown in the drawing for the removal of ticks comprises a main body 2 having a longitudinal recess 3 for accommodating a reservoir 4 for a freeze spray. Formed on the underside of the main body 2 is a transversely extending link section 5, extending through which is a channel 6 for the freeze spray, which opens into a discharge nozzle 7.

Formed on the underside of the recess 3 is an annular collar 8, against which a valve actuation element, which is not shown in the drawing, on the underside of the reservoir 4 is supported, so that when pressure is exerted onto the bottom 9 of the reservoir in the direction of the arrow 10, freeze spray is emitted.

Formed as one piece with the main body 2 is a forceps configuration 11 comprising two legs 12 that extend convexly arched outward from the upper side of the main body 2 along both sides of it into the region below the discharge nozzle 7.

Each leg 12 comprises an actuation section 13 having a ribbed surface 14, and a forceps section 15 incorporating one each forceps extension 16 extending inward from the forceps section 15, bent at right angle, at its underside.

Provided on the underside of the link section 5 is a transverse link 19, at the two outer ends of which bearing sections 20 are formed, into which a bearing extension 21 of the legs 12 engages that is formed between the actuation section 13 and forceps section 15.

Because of the pivot bearing 22 that is created in this manner, the exertion of pressure in the direction of the arrows 17 onto the actuation sections 13 results in the forceps extensions 16 being moved apart.

This means that when the device 1 is grasped with one hand on both sides of the legs 12, pressure can be exerted with the thumb or index finger in the direction of the arrow 10 onto the front end 9 of the reservoir 4, the legs 12 having first been pushed together inward in the direction of the arrows 17. This results in the forceps extensions 16 being moved apart, so that a gap 18, which is apparent in FIG. 2, is created that makes it possible to grasp around or underneath the tick's body in the region of the skin surface. When pressure is no longer exerted onto the actuation sections 13, the forceps sections 15 close, due to the elastic reset force of the legs 12. After the freezing process the tick's body can be removed from the skin effortlessly and without pain. As a result of the inventive design the freeze spray is aimed precisely onto the tick to be grasped. This results in a flash freezing, causing the tick to instantaneously become completely immobile, and all organs and the stomach content of the tick to become frozen. This prevents increased amounts of Borreliosis pathogens from being able to enter into the bite wound. The flash-frozen tick can be removed better and more easily.

What is claimed is:

1. A device for the removal of ticks from the skin of humans and animals comprising a forceps configuration for grasping the tick's body in the region of the skin surface, as well as a reservoir having an actuation means for a freeze spray to be sprayed onto the tick's body prior to its removal, wherein a main body (2) is provided that incorporates a hollow space (3) for accommodating the reservoir (4) for the defrosting [sic] spray, a discharge nozzle (7) for the freeze spray on the underside of the main body (2), and a forceps configuration ( 11) that extends around the main body (2) into the region below the discharge nozzle (7).

2. A device according to claim 1, wherein the main body (2) and forceps configuration (11) are injection-molded from plastic as one piece.

3. A device according to claim 1, wherein the forceps configuration (11) has two legs (12) that are symmetrical to each other, each leg (12) comprising an actuation section (13) and a forceps section (15), each leg (12) being convexly arched outward, and each leg (12) being pivot-mounted in the transition region between the actuation section (13) and forceps section (15) in such a way that when a force of pressure is exerted onto the actuation section (13) in an inward direction, the associated forceps section (15) is moved outward.

4. A device according to claim 3, wherein each pivot bearing (22) is formed at the outer end of a cross brace (19) disposed on the underside of the main body.

5. A device according to claim 4, wherein the two ends of the cross brace (19) have bearing openings (20) formed on them that are circular-segment shaped in cross-section, in which a bearing projection (21) of corresponding cross section can be locked into place that extends away from the inside of each leg (12) in the transition region of the actuation section (13) and grasping section (15).

6. A device according to claim 1, wherein the outsides of the actuation sections (13) have a ribbed surface (14).
